(19) United States
(12) Patent Application Publication
(10) Pub. No.: US 2025/0213269 A1
(43) Pub. Date: Jul. 3, 2025

(54) MESHER FOR THE EXPANSION OF SKIN GRAFTS

(71) Applicant: SIGN Fracture Care International, Richland, WA (US)

(72) Inventors: Randall J. Huebner, Portland, OR (US); Mary Louise Jensen, Richland, WA (US); Joel Gillard, Portland, OR (US)

(21) Appl. No.: 18/912,426

(22) Filed: Oct. 10, 2024

Related U.S. Application Data
(60) Provisional application No. 63/589,295, filed on Oct. 10, 2023.

(51) Int. Cl. A61B 17/322; A61B 17/00

(52) U.S. Cl. CPC A61B 17/322; A61B 2017/00747; A61B 2017/3225

(57) ABSTRACT

A mesher for creating a repeating pattern of slits in a skin graft. The mesher may include (1) a bottom plate having a support surface that engages the skin graft from below; (2) a top plate having a holding surface that fits over the bottom plate and engages the skin graft from above; and a registration element shared between the top and bottom plates that sets and maintains a relative alignment of the plates. The support surface may include an array of recesses defined by a column spacing and a row spacing. The holding surface may include a plurality of parallel slots separated laterally by the column spacing and sized to receive a cutting blade used to perform the meshing. Finally, the registration element may align the slots with respective columns of recesses.

### CROSS-REFERENCES TO RELATED APPLICATION

This application is based upon and claims the benefit under 35 U.S.C. § 119(e) of U.S. Provisional Patent Application Ser. No. 63/589,295, filed Oct. 10, 2023, which is incorporated herein by reference in its entirety for all purposes.

### INTRODUCTION

Skin grafts may be used to treat open wounds, surgical skin excisions, burns, and other skin lesions. Typically, an autograft of healthy skin is harvested from a donor site on a patient and then used to cover a lesion at another site on the same patient. Large skin grafts may be “meshed” by cutting holes or slits in them before they are placed over the lesion. Meshing allows greater coverage because the meshed skin graft may be stretched to cover a larger area than the original graft. Meshing also promotes healing because the openings created by meshing provide gaps that allow the transfer of fluids from the wound bed during healing. Unfortunately, current meshers are complicated, expensive, and difficult to clean. Thus, there is a need for a simpler, less expensive mesher, especially for use in poorer or more poorly equipped operating rooms.

### SUMMARY

The present disclosure provides a mesher for creating a repeating pattern of slits in a skin graft. The mesher may include (1) a bottom plate having a support surface that engages the skin graft from below; (2) a top plate having a holding surface that fits over the bottom plate and engages the skin graft from above; and a registration element shared between the top and bottom plates that sets and maintains a relative alignment of the plates. The support surface may include an array of recesses defined by a column spacing and a row spacing. The holding surface may include a plurality of parallel slots separated laterally by the column spacing and sized to receive a cutting blade used to perform the meshing. Finally, the registration element may align the slots with respective columns of recesses.

### BRIEF DESCRIPTION OF THE DRAWINGS

FIG. 1 is a top view of the bottom plate of a mesher showing a support surface having a pattern of recesses onto which a skin graft may be placed.

FIG. 2 is a top view of a top plate of a mesher showing a plurality of slots for receiving a cutting blade.

FIG. 3 is an isometric view of a skin mesher formed by the assembly of the bottom and top plates of FIGS. 1 and 2, respectively.

FIG. 4 is a cross-sectional view of the mesher of FIG. 3, taken generally along the long direction of the slots in FIG. 3, showing the assembly of the bottom and top plates of the mesher with a skin graft sandwiched between.

FIG. 5 is a perspective view of the mesher of FIGS. 3 and 4 showing how a cutting blade can be inserted into a slot of the top plate to mesh a skin graft.

FIG. 6 is a top view of a skin graft resting on a bottom plate before meshing (left) and after meshing and subsequent expansion (right).

FIG. 7 is a side-by-side pair of top views of mesher bottom plates, showing respective support surfaces, patterns of recesses, and alignment features. The bottom plate in Panel A is used to create meshed grafts with a 3:1 expansion ratio. The bottom plate in Panel B is used to create meshed grafts with a 2:1 expansion ratio.

FIG. 8 is a top view of a mesher top plate, suitable for use with the bottom plates of FIGS. 1 and 7, showing a plurality of slots and indicia used to denote and distinguish slots.

### DETAILED DESCRIPTION

The present disclosure provides a mesher for creating a repeating pattern of slits in a skin graft. The mesher may include (1) a bottom plate having a support surface that engages the skin graft from below; (2) a top plate having a holding surface that fits over the bottom plate and engages the skin graft from above; and a registration element shared between the top and bottom plates that sets and maintains a relative alignment of the plates. The support surface may include an array of recesses defined by a column spacing and a row spacing. The holding surface may include a plurality of parallel slots separated laterally by the column spacing and sized to receive a cutting blade. Finally, the registration element may align the slots with respective columns of recesses. A repeating pattern of slits in the skin graft may be created by positioning the skin graft between the bottom and top plates of the mesher, inserting a cutting blade into a slot of the top plate until it engages the skin graft, pulling the cutting blade through a length of the slot such that it preferentially cuts the graft between the recesses, and repeating the process of inserting and pulling for at least one additional slot. The pattern of slits cut in the skin is determined by the size and spacing of the recesses and slots. Exemplary expansion ratios may range from 1:1 to 6:1, among others. The disclosure further provides kits in which the mesher is combined with at least one of a dermatome, an additional bottom plate, and a cutting blade. Finally, the disclosure provides methods for using both the mesher and the kits. Further aspects of the system are described below, beginning with figures showing the bottom and top plates and how they can be assembled to form the mesher, and finishing with a set of examples discussing how the mesher can be used to form a meshed skin graft.

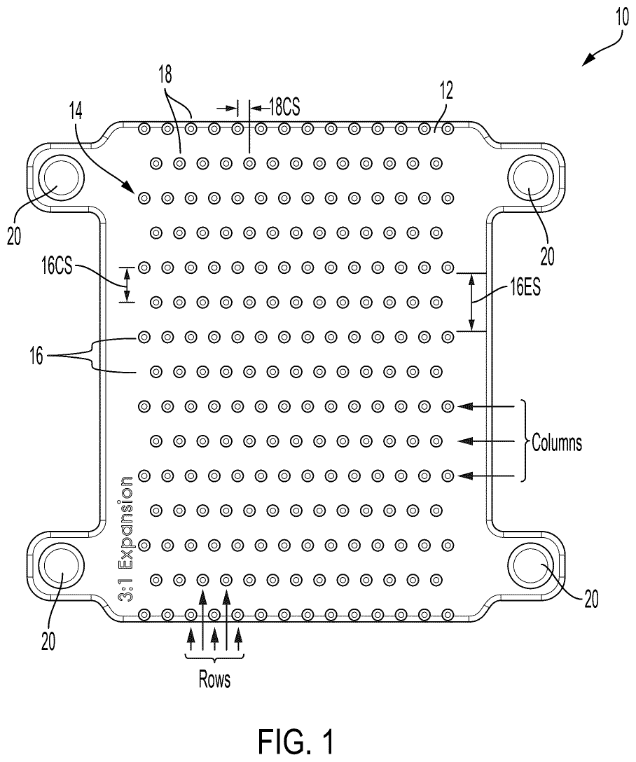

FIG. 1 shows an exemplary bottom plate 10 for use in a mesher. The bottom plate includes a support surface 12 on which a skin graft can be placed for meshing. The support surface may include a pattern of recesses 14, comprising indentations, holes, pits, and/or other depressions in the surface, which will lie below the skin graft. Here, the recesses are evenly spaced in an array of columns 16 and rows 18 characterized by a column spacing 16S and a row spacing 18S, respectively. The recesses and array may have any suitable absolute and relative sizes. For example, the recesses may be about 3 mm in diameter, the column spacing may be about 15 mm to 20 mm, and the row spacing may be about 3.2 mm, among others. The bottom plate further may include registration elements, such as posts 20, to align the recesses of the bottom plate with features, such as apertures, of the top plate of a mesher. The posts may function as support legs for two-sided bottom plates. Specifically, for bottom plates having two oppositely disposed support surfaces and oppositely disposed posts, the posts opposite the support surface being used to engage the skin graft may function as supports, while the posts adjacent the support surface being used to engage the skin graft may function as registration features. The expansion ratios for recess patterns on both sides of a two-sided bottom plate may be the same or different.

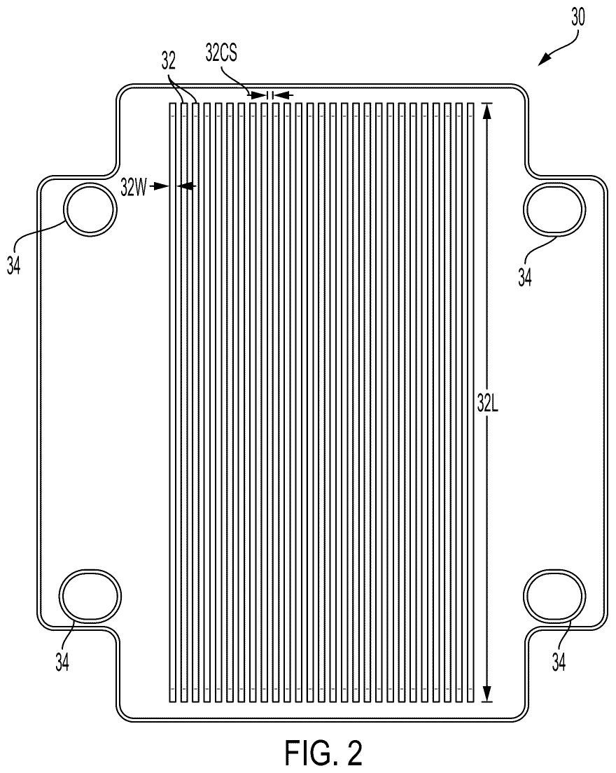

FIG. 2 shows an exemplary top plate for use in a mesher. The top plate includes a plurality of parallel slots 32 extending through the plate with a width 32W (such as about 1 mm) chosen to allow insertion of a cutting blade and a length 32L that matches or exceeds the length of the bottom plate in FIG. 1. The slot-to-slot slot spacing 32S matches the row-to-row recess spacing 18S in FIG. 1. The top plate may further include registration elements, such as apertures 34, to align the top plate with the bottom plate and simultaneously align the slots and recesses in the two plates. Apertures 34 may have any suitable shape(s), including circular, oval, or a combination of both, among other possibilities.

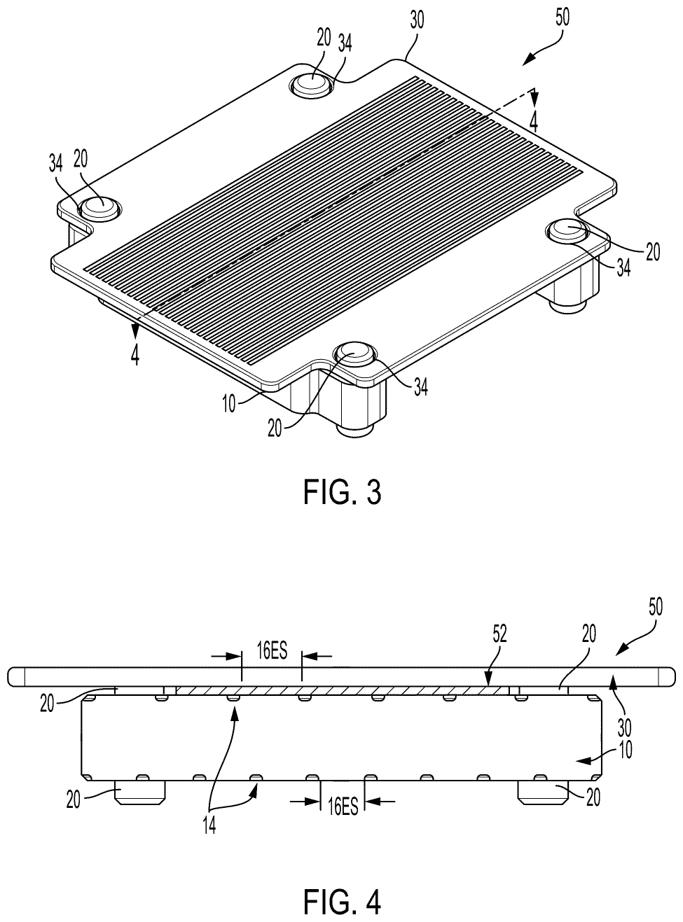

FIG. 3 shows an exemplary assembled mesher 50 formed by pairing the bottom plate 10 of FIG. 1 with the top plate 30 of FIG. 2. The registration elements (apertures) 34 in the top plate and associated alignment features (posts) 20 in the bottom plate ensure that the rows 18 of recesses in the bottom plate are in line with the slots 32 of the top plate. The harvested skin graft is placed between the plates for meshing.

FIG. 4 shows a sectional view of the mesher of FIG. 3, taken generally along the line 4-4 (i.e., in a direction parallel to the slots) in FIG. 3. Here, a skin graft 52 is sandwiched between the top plate 30 and bottom plate 10. A row of recesses 14 and an individual slot 32 are aligned. The edge-to-edge distance 18ES between recesses in the row defines the length of the cut slit in the skin graft because the skin flexes into each recess and so is uncut at those points during meshing. The pairs of adjacent recesses in FIG. 4 are separated by two columns of recesses in the transverse direction (see FIG. 1). The bottom plate in FIG. 4 has two support surfaces, each independently having the same or a different meshing ratio, such as 2:1 on the top surface and 3:1 on the bottom surface, among others. The different meshing ratios reflect different edge-to-edge spacings 16ES, 16ES' between recesses in the top and bottom surfaces of the bottom plate. Posts 20 act as supports when facing down and registration features (that engage with the top plate) when facing up. In other embodiments, only one support surface (the one that receives the skin graft and that contacts the top plate) may have recesses.

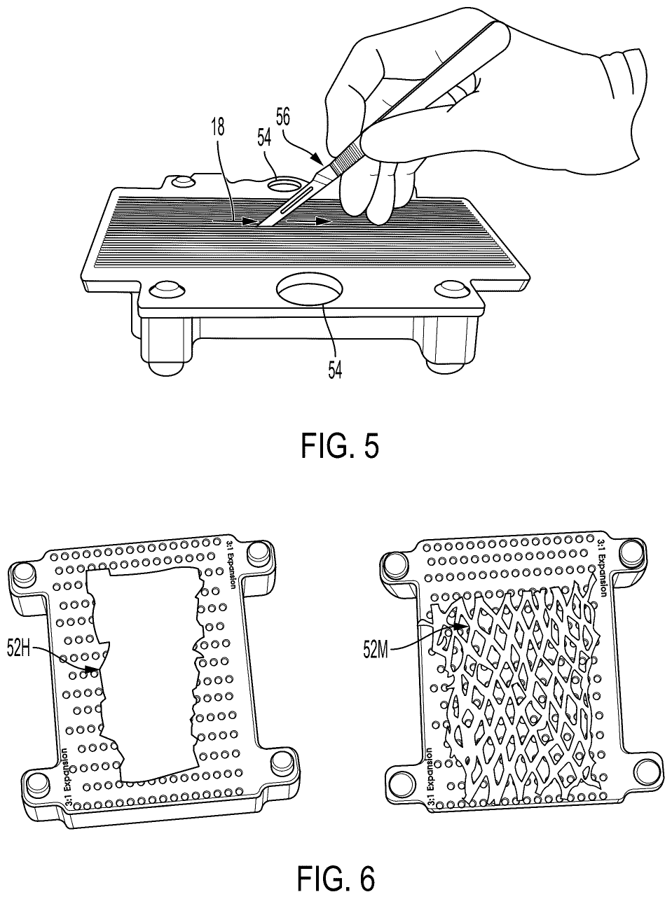

FIG. 5 shows the mesher 50 of FIGS. 3 and 4 in use. Here, the mesher top plate 30 includes optional handle features 54 that may be used to hold and place the top plate over the skin graft and bottom plate. A cutting blade 54 is inserted into the top of a slot in the top plate and advanced (in the pictured case) toward the user, as shown by the arrows, for the entire length of the slot or a desired portion thereof. The cutting blade can be any device capable of fitting into the slot and cutting the skin graft. Examples include, among others, a typical scalpel having a blade size of number 10 or larger and a circular rotary blade of 35 mm in diameter or larger. The process is repeated for most or all of the slots in the top plate, allowing most or all of the skin graft to be meshed, as desired. The skin graft is serially cut with a pattern of slits equal in length to the distance 20 between recesses, i.e., recess column spacing 11. This happens because the flexibility of skin allows the skin graft to flex into the recesses, such that the skin graft will not be cut by the blade when advancing over a recess. In some cases, more than one cutting blade may be used simultaneously, for example, by holding two or three (or more) scalpels next to one another in distinct (e.g., adjacent) slots. Alternatively, a special tool may be used with two or more blades, spaced apart by the slot-to-slot spacing, sharing a single handle.

FIG. 6 shows (at left) the as-harvested (original) skin graft 52H placed onto the bottom plate prior to meshing and (at right) the meshed skin graft 52W, including a pattern of diamond-shaped slits with graft expansion. Following meshing, the skin graft can be transplanted over a wound defect of the patient. The meshed graft will cover a larger area than an unmeshed graft, using less donor site skin, while facilitating the transfer of fluids during healing.

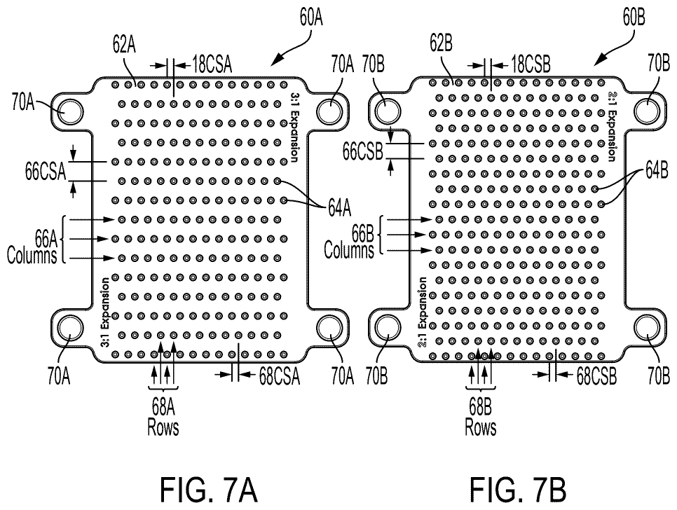

FIG. 7 shows exemplary mesher bottom plates 60A,B, which are similar to bottom plate 10 in FIG. 1, emphasizing respective support surfaces 62A,B, patterns of recesses 64A,B, and registration elements 70A,B. Bottom plates 60A,B may be used to create meshed skin grafts with expansion ratios of 3:1 (Panel A) and 2:1 (Panel B), respectively. Both bottom plates include columns 66A,B and rows 68A,B. The center-to-center row-to-row spacing 68CSA,CSAB is the same in both bottom plates, which allows the same top plate to be used with both bottom plates (because the slots in the top plate can align with the rows of recesses in both bottom plates). However, the center-to-center column-to-column spacing 68CSA,68CSB is different between the two bottom plates. It is this difference that leads to different expansion ratios when the plates are used to mesh a skin graft. Specifically, larger spacings, such as those in the bottom plate of Panel A, correspond to longer cuts and higher expansion ratios in the meshed graft. Conversely, smaller spacings, such as those in the bottom plate of Panel B, correspond to shorter cuts and lower expansion ratios. Here, both plates have the same number (27) of rows of recesses; however, they have different numbers (15 vs 19) of columns of recesses. The bottom plate, more generally, may have any suitable numbers of columns and rows and of recesses in each. These two bottom plates may from completely separate meshers, may be on flip sides of the bottom plate of the same mesher, and/or may be combined with yet other recess patters corresponding to expansion ratios,

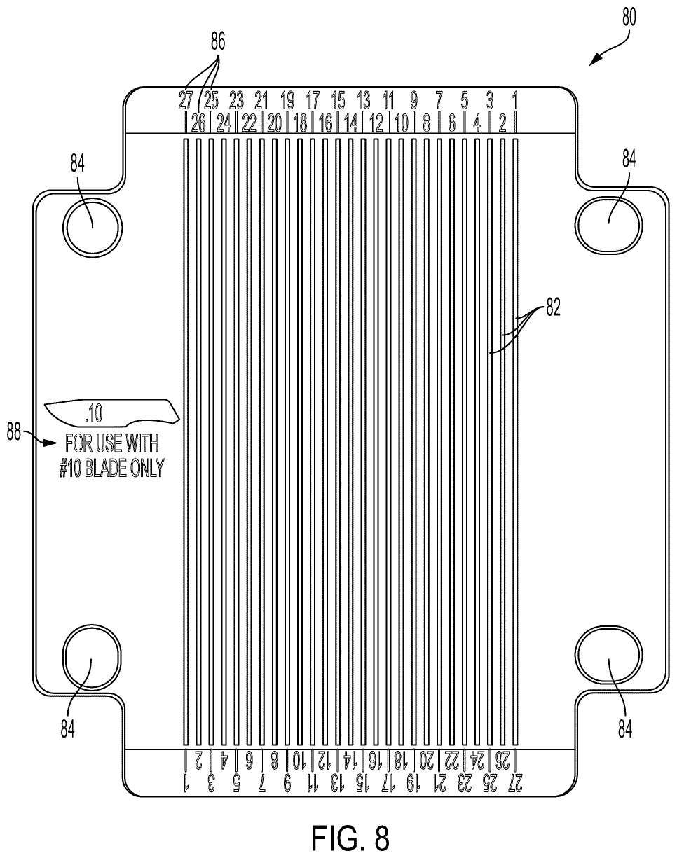

FIG. 8 shows a top view of a mesher top plate 80, similar to top plate 30 in FIG. 2 and suitable for use with bottom plates 10 and 60A,B of FIGS. 1 and 7, respectively, showing a plurality of slots 82, registration elements 84, and indicia 86. The slots can be used to receive and guide a cutting blade for meshing a skin graft. The registration elements can be used to align the top plate with a corresponding bottom plate, so that slots and recesses on the top plate and bottom plate, respectively, are in registration. The indicia, which may include numbers, letters, symbols, and/or other markers capable of distinguishing slots, can be used to reduce the likelihood that slots are missed and/or double-cut during meshing. The top plate may further include warnings, reminders, and/or other text and/or graphics, at one or more locations, such as a warning to use only a #10 blade for cutting.

The bottom and top plates may be formed from any suitable material(s). Examples include a biocompatible material, such as a polymer or metal, that can be sterilized, that can safely contact human tissue, and that is suitable (in the case of the bottom plate) as a cutting surface. For example, among other possibilities, the bottom plate may be formed of a polymer to facilitate cutting, while the top plate may be formed of a metal to guide reproducible cutting blade motion. The top plate may have sufficient weight to hold the skin graft in place during meshing.

The bottom and top plates, and thus the mesher, may have any suitable dimensions. In some cases, the skin graft may be up to about five inches long (L) and about three inches wide (W). Accordingly, among other possibilities, the bottom plate may be about 5.5 inches long and about 6 inches wide, while the top plate may be about 7 inches long and about 6.25 inches wide.

### EXAMPLES

The following examples describe further aspects of the present disclosure, including a skin mesher kit, methods for skin graft harvesting and skin graft meshing, cautions and advisories, troubleshooting, and selected embodiments. The examples are intended for illustration and should not be interpreted as limiting the entire scope of the present disclosure. Each example may include one or more distinct embodiments and/or contextual or related information.

### Example 1: Skin Mesher Kit

This example describes an exemplary kit for harvesting and meshing a skin graft, in accordance with aspects of the present disclosure.

The exemplary kit may include a skin mesher, as disclosed herein, and one or more of the following: a dermatome for harvesting a skin graft, an additional bottom plate for the skin mesher, and a cutting blade. The dermatome optionally may consist of one or more parts, including a handle assembly, a blade, and a cap assembly for joining and securing the three parts. In some embodiments, the kit may include additional replacement blades for the dermatome. The cutting blade may include any instrument suitable for cutting the skin graft while the graft is positioned in the skin mesher. Suitable cutting blades include scalpels, such as a #10 scalpel, and/or a rotary blade, among others. The kit further may include a tray suitable for housing, organizing, and/or protecting the functional components of the kit. Components of the kit may be formed of any suitable materials. For example, the dermatome, mesher top plate, scalpel, and some or all of the tray may be formed of stainless steel. The mesher bottom plate may be formed of a softer material, such as a polymer, among others. Additional materials, especially for the tray, may include aluminum and/or polymer, among others. Components of the kit may be designed to be steam sterilized (e.g., autoclaved) and reused for multiple cases.

The exemplary kit may be used to create and mesh skin grafts with sizes consistent with the size of the dermatome and mesher for any suitable indications. For example, the kit may be suitable for traumatic wound coverage, with a dermatome capable of harvesting skin grafts with widths up to about 50 mm and split thicknesses of about 0.3 mm to about 0.6 mm, and a mesher capable of graft expansion ratios of 2:1 and 3:1, among others.

### Example 2: Skin Graft Harvesting

This example describes an exemplary method for harvesting a skin graft for use with the skin mesher of the present disclosure. Step 1: Select a harvest site for the skin graft. Step 2: Outline the desired boundaries of the skin graft. The boundaries will set the size and shape of the graft. Keep in mind (A) that a flat firm area will provide the best quality graft, (B) that the width of the unmeshed graft will at least approximately match the width of the dermatome (skin graft knife), and (C) the expansion ratios of the mesher (e.g., about 2:1, about 3:1, etc.). Step 3: Remove hair from the harvest site. Step 4: Lubricate the skin with a suitable lubricant, such as mineral oil or liquid soap, such as chlorhexidine, among others. Step 5: Tension and flatten the skin at the harvest site, for example, using a stiff board, a flat palm with a dry lap sponge to retract the skin, and/or distal traction for patients with loose skin or obesity. Step 6: Harvest the skin graft using the dermatome and/or other instrument. This may involve (A) holding the dermatome at a suitable angle (e.g., about 20 degrees) relative to the skin surface, (B) applying downward pressure against the skin, and/or (C) oscillating the dermatome side-to-side while advancing forward during the cut. The depth of the cut, and thus the thickness of the graft, may be controlled by adjusting the angle (A) and pressure (B) of the dermatome. For example, decreasing the angle will lead to a thinner graft, and increasing the angle will lead to a thicker graft. Step 7: Keep the harvested skin graft moist prior to meshing, for example, by placing it onto or between saline-soaked gauze after harvesting.

### Example 3: Skin Graft Meshing

This example describes an exemplary method for meshing a skin graft, in accordance with aspects of the present disclosure. Step 1: Select a skin mesher having top and bottom plates, as described in the present disclosure. Step 2: Place the bottom plate of the skin mesher on a stable surface, with the up side corresponding to a desired expansion ratio (e.g., about 2:1, about 3:1, etc.). Step 3: Place the skin graft onto the up side of the bottom plate, flattening and uncurling the graft, as needed. Step 4: Place the top plate of the skin mesher over the skin graft and bottom plate, aligning the holes in the top plate with the posts in the bottom plate. Step 5: Insert a cutting blade, or a plurality of cutting blades, into a corresponding number of slots in the top plate. Typically, the cutting blade(s) will initially be placed in slots near a side of the top surface to facilitate methodical cutting. A suitable cutting blade may be a #10 scalpel. Step 6: Hold the cutting blade(s) at a 30-45 degree angle relative to the horizontal. Step 7: Cut the skin graft by pulling the cutting blade(s) through the slot, typically toward the person making the cut. This may involve (A) holding the cutting blade(s) at a suitable angle (e.g., about 30-45 degrees from the horizontal), and (B) applying downward pressure against the skin graft. Cutting at too low (shallow) or high (steep) an angle, and/or using a smaller size cutting blade (e.g., a #15 scalpel) may elongate and connect cuts in the skin graft. The amount of applied pressure should be similar to that used to make an incision. Too little pressure may not cut the skin graft. Too much pressure will damage the mesher plates over multiple uses. Step 8: Repeat the process by cutting through every slot over the graft. Markings on the top plate and/or a properly placed finger can be used to keep track of slots. Step 9: Remove the meshed skin graft, and prepare the graft for placement on a wound (e.g., by pulling on sides of the graft parallel to the cut direction to expand the graft). Rinse the graft with a saline solution prior to placement on a wound.

### Example 4: Cautions and Advisories

This example describes exemplary cautions and advisories for consideration in connection with the skin mesher of the present disclosure.

Blade sharpness. It is desirable to use clean, sharp cutting tools when preparing skin grafts. Dull blades, which can arise from repeated use, may make it difficult to harvest consistent grafts and meshes. These difficulties can be avoided by using a new sharp blade with each patient or, if cleaning is needed, using cleaning techniques that protect blade sharpness (e.g., steam sterilization). On the flip side, blades in general, and sharp blades in particular, can cut users if not handled properly. Therefore, care should be taken with blades used for skin harvesting and skin meshing, particularly when loading and unloading dermatome blades (if replaceable) and meshing the graft with the cutting tool (e.g., scalpel).

Skin graft thickness. The thickness of the skin graft may be controlled during harvesting by the angle of the dermatome relative to the skin, the pressure applied to the dermatome, and the tension of the skin. Typical grafts may be between about 0.3 mm to about 0.6 mm thick, among others. It may be difficult to obtain split thickness grafts with some dermatomes, particularly with young children (e.g., under age five), the elderly, and/or others having thin atrophic dermis.

Wear and tear. Wear and tear, and the accumulation of debris, can occur over time with both the dermatome and mesher plates. These components should be inspected for signs of wear and/or other problems prior to each use. Wear and tear can be reduced by using the minimum amount of pressure necessary during cutting necessary to achieve graft thickness and meshing. The system may be manufactured and distributed such that worn parts are replaceable.

Indications. The size of the mesher may influence or determine the uses to which skin grafts may be put. For example, smaller meshers may be more suitable for traumatic wound coverage and may not harvest or sufficiently expand grafts for burn coverage. Therefore, appropriate graft pre-planning may be needed.

### Example 5: Troubleshooting

This example describes exemplary troubleshooting tips that may be used, if at all, to address selected performance issues that may arise with the system and/or user technique. First, if meshing areas are incompletely cut or unmeshed, it may be advisable to recut affected portions of the graft with a scalpel or other cutting tool without using the top plate to avoid difficulties inherent in trying to realign the partially cut graft to the slots. Second, if the harvested graft has holes or ragged edges, it may be advisable to adjust which slots are used during meshing to avoid the compromised areas. Third, if the skin graft is too thick or too thin, it may be advisable to watch the donor bed during harvest. In this way, if small areas of yellow adipose tissue are present, suggesting that the graft is too thick, the downward pressure and angle of the dermatome may be adjusted for a shallower graft. Alternatively, if holes are created during harvest, suggesting that the graft is too thin, the downward pressure and dermatome angle may be adjusted for a deeper graft. Fourth, if the plate and/or support posts have suitable symmetry axes, it may be possible to install the mesher top plate in different orientations. For example, the top plate in FIGS. 1-6 may be installed with either side up. Moreover, in both cases, the top plate may be installed in four different orientations about a perpendicular, each separated by 90 degrees. Some or all of these different orientations may be degenerate. However, the markings on different sides may vary.

### Example 6: Selected Aspects

This example describes additional selected aspects of the present disclosure, presented without limitation as a series of paragraphs, some or all of which may be numerically indexed for clarity and efficiency. Each of these paragraphs can be combined with one or more other paragraphs, and/or with disclosure from elsewhere in this application, in any suitable manner. Many of the indexed paragraphs expressly refer to and further limit other indexed paragraphs, providing without limitation examples of some of the suitable combinations.

1. A mesher for creating a repeating pattern of slits in a skin graft, comprising (A) a bottom plate having a support surface that engages the skin graft from below; (B) a top plate having a holding surface that engages the skin graft from above; and (C) a registration element shared between the top and bottom plates that sets and maintains a relative alignment of the plates; wherein the support surface includes an array of recesses defined by a column spacing and a row spacing, wherein the holding surface includes a plurality of parallel slots separated laterally by the row spacing in the support surface and sized to receive a cutting blade, and wherein the registration element aligns the slots with respective rows of recesses; and wherein the repeating pattern of slits in the skin graft is created by positioning the skin graft between the bottom and top plates, inserting a cutting blade into a slot of the top plate until it engages the skin graft, pulling the cutting blade through a length of the slot such that it preferentially cuts the graft between the recesses, and repeating the process of inserting and pulling for at least one additional slot.

2. The mesher of paragraph 1, wherein a spacing between adjacent recesses in a given column of recesses in the bottom plate is an integral multiple of a spacing between adjacent slots in the top plate.

3. The mesher of paragraph 2, wherein the integral multiple is two.

4. The mesher of any preceding paragraph, wherein there are about twice as many rows of recesses as there are recesses in a column of recesses, for example, twenty-seven rows of recesses and either thirteen or fourteen recesses in each column, or fifteen rows of recesses and either seven or eight recesses in each column, among other possibilities.

5. The mesher of any preceding paragraph, wherein there is a row of recesses in the bottom plate for each slot in the top plate.

6. The mesher of any preceding paragraph, the recesses having a diameter in the plane of the support surface, wherein the diameter of the recesses is at least approximately equal to a lateral separation between slots in the top plate.

7. The mesher of paragraph 6, wherein the diameter of the recesses and the lateral separation between slots are both about 3 mm.

8. The mesher of any preceding paragraph, wherein a spacing between adjacent columns of recesses is equal to or greater than a spacing between adjacent rows of recesses.

9. The mesher of paragraph 8, wherein the spacing between adjacent columns of recesses is greater than the spacing between adjacent rows of recesses.

10. The mesher of any preceding paragraph, wherein recesses in immediately adjacent rows and columns are offset from one another.

11. The mesher of any preceding paragraph, wherein the recesses have a column spacing of 15 mm to 20 mm.

12. The mesher of any preceding paragraph, wherein meshing the skin graft allows the skin graft to be expanded, and wherein the expansion ratio between the expanded skin graft and the original (unmeshed) skin graft is greater than 1:1 and less than 6:1.

13. The mesher of any preceding paragraph, wherein the registration element includes mating features in the top and bottom plates.

14. The mesher of paragraph 13, wherein the mating features include a post in the bottom plate and a corresponding aperture configured to receive the post in the top plate.

15. The mesher of paragraph 14, wherein the mating features include four posts in the bottom plate and a corresponding four apertures in the top plate.

16. The mesher of paragraph 15, the bottom and top plates being at least substantially rectangular with four corners, wherein the posts and apertures are respectively positioned at or adjacent the four corners.

17. The mesher of paragraph 16, wherein the bottom plate includes pairs of opposed posts that can function as supports for the mesher when turned down and as mating features for receiving the apertures in the top plate when turned up.

18. The mesher of any preceding paragraph, wherein the recesses and slots are precisely located relative to the registration element.

19. The mesher of any preceding paragraph, wherein the bottom plate includes an at least substantially planar slab portion having opposed surfaces, and wherein each opposed surface includes an array of recesses and can function as a support surface for engaging a skin graft when turned up to receive the top plate.

20. The mesher of paragraph 19, wherein a geometry of the array of recesses on each surface is different, such that the expansion ratios for skin grafts meshed on each surface is different.

21. The mesher of paragraph 20, wherein the expansion ratio for skin grafts meshed on one of the opposed surfaces is 2:1 and the expansion ratio for skin grafts meshed on the other of the opposed surfaces is 3:1.

22. The mesher of any preceding paragraph, wherein the slots are sized to accept at least one of a size 10 or larger scalpel and a size 35-mm diameter or larger circular blade.

23. The mesher of any preceding paragraph, wherein the mesher is sized to receive and mesh skin grafts of at least about 3 by 5 inches.

24. The mesher of any preceding paragraph, the top and bottom plates each being characterized by a respective lateral dimension, wherein the lateral dimension for each is at least about 10 cm.

25. The mesher of paragraph 24, wherein the lateral dimension (and/or an area) of the top plate is larger than the lateral dimension (and/or an area) of the bottom plate.

26. The mesher of any preceding paragraph, wherein the mass of the top plate is sufficient to allow the top plate to apply pressure to the skin graft that holds the graft in place during meshing.

27. The mesher of paragraph 26, wherein the mass of the top plate is about 1 kg.

28. The mesher of any preceding paragraph, wherein the slots are uniquely indexed for the user to keep track of which slots have been used with a cutting blade.

29. The mesher of paragraph 28, wherein the slots are uniquely indexed with a number, a letter, or both.

30. The mesher of any preceding paragraph, wherein the bottom plate and top plate comprise biocompatible materials safe for contacting human tissue.

31. The mesher of any preceding paragraph, wherein the bottom plate and top plate can be sterilized and reused.

32. The mesher of any preceding paragraph, wherein the bottom plate comprises a polymer and the top plate comprises a metal.

33. The mesher of any preceding paragraph, wherein the top plate includes at least one handle feature to facilitate placing the top plate over the bottom plate before meshing and removing the top plate from the bottom plate after meshing.

34. The mesher of paragraph 33, wherein the handle feature includes at least one hole in the plate.

35. A kit for meshing a skin graft, comprising (A) the mesher of any of paragraphs 1-34; and (B) at least one of a dermatome, an additional bottom plate, and a cutting blade.

36. The kit of paragraph 35, further comprising at least two of a dermatome, an additional bottom plate, and a cutting blade.

37 The kit of paragraph 36, further comprising a dermatome, an additional bottom plate, and a cutting blade.

38. A method of creating a meshed skin graft, comprising (A) harvesting a section of tissue from a patient; (B) placing the graft between the bottom plate and top plate of the mesher of any of paragraphs 1-33; (C) inserting a cutting blade through a slot in the top plate and against the bottom plate; and (D) pulling the cutting blade with a downward pressure along the slot for the length of the graft.

39. The method of paragraph 38, further comprising repeating the steps of inserting and pulling for additional slots.

40. The method of paragraph 38 or 39, the mesher having two opposed support surfaces configured to produce different expansion ratios, further comprising selecting one of the two opposed surfaces and its respective expansion ratio prior to the step of placing the graft.

41. The method of any of paragraphs 38-40, the skin graft being a first skin graft, further comprising sterilizing the top and bottom plates and repeating the steps of harvesting, placing, inserting, and pulling with a second skin graft.

42. The method of paragraph 41, wherein the first skin graft is meshed with a first expansion ratio using a first surface of the bottom plate, and wherein the second skin graft is meshed with a second expansion ratio using a second (opposing) surface of the bottom plate.

The term “and/or” as used in the present disclosure means all combinations of the listed elements. For example, a list with two elements “A and/or B” covers three possibilities: only A, only B, and both (A and B). Similarly, a list with three elements “A, B, and/or C” covers seven possibilities: only A, only B, only C, both A and B, both A and C, both B and C, and all three (A, B, and C). The extension to four or more elements follows the same pattern.

The term “exemplary” as used in the present disclosure means “illustrative” or “serving as an example” and is not intended to imply desirability or superiority.

The terms “first,” “second,” “A,” “B,” and so on are used in the present disclosure to distinguish or identify various members of a group, or the like, in the order in which they are introduced in a particular context and are not intended to show serial or numerical limitation.

The disclosure set forth herein may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

What is claimed:

1. A mesher for creating a repeating pattern of slits in a skin graft, comprising:
a bottom plate having a support surface that engages the skin graft from below;
a top plate having a holding surface that engages the skin graft from above; and
a registration element shared between the top and bottom plates that sets and maintains a relative alignment of the plates;
wherein the support surface includes an array of recesses defined by a column spacing and a row spacing, wherein the holding surface includes a plurality of parallel slots separated laterally by the column spacing in the support surface and sized to receive a cutting blade, and wherein the registration element aligns the slots with respective columns of recesses; and
wherein the repeating pattern of slits in the skin graft is created by positioning the skin graft between the bottom and top plates, inserting a cutting blade into a slot of the top plate until it engages the skin graft, pulling the cutting blade through a length of the slot such that it preferentially cuts the graft between the recesses, and repeating the process of inserting and pulling for at least one additional slot.

2. The mesher of claim 1, wherein a spacing between adjacent recesses in a given row of recesses in the bottom plate is an integral multiple of a spacing between adjacent slots in the top plate.

3. The mesher of claim 1, wherein there are about twice as many rows of recesses as there are recesses in a column of recesses.

4. The mesher of claim 1, wherein there is a column of recesses in the bottom plate for each slot in the top plate.

5. The mesher of claim 1, the recesses having a diameter in the plane of the support surface, wherein the diameter of the recesses is at least approximately equal to a lateral separation between slots in the top plate.

6. The mesher of claim 1, wherein recesses in immediately adjacent rows and columns are offset from one another.

7. The mesher of claim 1, wherein the registration element includes mating features in the top and bottom plates.

8. The mesher of claim 7, wherein the mating features include a post in the bottom plate and a corresponding aperture configured to receive the post in the top plate.

9. The mesher of claim 8, the bottom and top plates being at least substantially rectangular with four corners, wherein mating features include four posts in the bottom plate and a corresponding four apertures in the top plate, and wherein the posts and apertures are respectively positioned at or adjacent the four corners.

10. The mesher of claim 9, wherein the bottom plate includes pairs of opposed posts that can function as supports for the mesher when turned down and as mating features for receiving the apertures in the top plate when turned up.

11. The mesher of claim 1, wherein the recesses and slots are precisely located relative to the registration element.

12. The mesher of claim 1, wherein the bottom plate includes an at least substantially planar slab portion having opposed surfaces, and wherein each opposed surface includes an array of recesses and can function as a support surface for engaging a skin graft when turned up to receive the top plate.

13. The mesher of claim 12, wherein a geometry of the array of recesses on each surface is different, such that the expansion ratios for skin grafts meshed on each surface is different.

14. The mesher of claim 1, wherein an area of the top plate is larger than an area of the bottom plate.

15. The mesher of claim 1, wherein the mass of the top plate is sufficient to allow the top plate to apply pressure to the skin graft that holds the graft in place during meshing.

16. The mesher of claim 1, wherein the slots are uniquely indexed for the user to keep track of which slots have been used with a cutting blade.

17. The mesher of claim 1, wherein the bottom plate comprises a polymer and the top plate comprises a metal.

18. The mesher of claim 1, wherein the top plate includes at least one handle feature to facilitate placing the top plate over the bottom plate before meshing and removing the top plate from the bottom plate after meshing.

19. A kit for meshing a skin graft, comprising:
the mesher of claim 1; and
at least one of a dermatome, an additional bottom plate, and a cutting blade.

20. A method of creating a meshed skin graft, comprising:
harvesting a section of tissue from a patient;
placing the graft between the bottom plate and top plate of the mesher of claim 1;
inserting a cutting blade through a slot in the top plate and against the bottom plate;
pulling the cutting blade with a downward pressure along the slot for the length of the graft; and
repeating the steps of inserting and pulling for additional slots.
